(19) United States
(12) Patent Application Publication
(10) Pub. No.: US 2025/0235325 A1
(43) Pub. Date: Jul. 24, 2025

(54) HEIGHT-ADJUSTABLE SPINAL FUSION CAGE

(71) Applicants: L&K BIOMED CO., LTD., Yongin-si (KR); Gook Jin KANG, Seoul (KR)

(72) Inventors: Gook Jin KANG, Seoul (KR); Youngbo AHN, Irvine, CA (US); Sang Soo LEE, Anyang-si (KR); Sun Gak CHOI, Gwangju-si (KR)

(73) Assignee: L&K BIOMED CO., LTD., Yongin-si (KR)

(21) Appl. No.: 19/173,322

(22) Filed: Apr. 8, 2025

Related U.S. Application Data
(63) Continuation of application No. 17/912,709, filed on Sep. 19, 2022.

(30) Foreign Application Priority Data
Mar. 20, 2020 (KR) 10-2020-0034675

(51) Int. Cl. A61F 2/44; A61F 2/30

(52) U.S. Cl. CPC A61F 2/447; A61F 2002/30324; A61F 2002/3055; A61F 2002/30579

(57) ABSTRACT

The present invention relates to a spinal fusion cage which is inserted between vertebral bodies in a state where the cage has the lowest height and is height-adjustable in the inserted state, thus making it possible to replace cages having heights in a certain range by a single cage. Therefore, manufacturers can reduce product groups that need to be produced and can also reduce product stock. Further, in contrast to the conventional cages having predetermined heights at regular intervals, the height of the inventive cage can be linearly adjusted according to the distance between the vertebral bodies of a patient, and thus a surgery for the patient can be performed using the cage adjusted to an optimum height according to the patient's condition.

### CROSS REFERENCE TO RELATED APPLICATIONS

This application is a continuation application of Ser. No. 17/912,709 filed Sep. 9, 2022, which is a National Stage of International Application No. PCT/KR2020/015473 filed Nov. 6, 2020, claiming priority based on Korean Patent Application No. 10-2020-0034675 filed Mar. 20, 2020.

### TECHNICAL FIELD

The present invention relates to a height-adjustable spinal fusion cage. More specifically, the present invention relates to a height-adjustable spinal fusion cage which is inserted between vertebral bodies in a state where the cage has the lowest height, and is height-adjustable in the inserted state.

### BACKGROUND ART

The vertebral body includes 32 to 35 vertebrae and intervertebral disks, which are simply called disks, between the vertebrae, and is the central part of the body that connects the skull at the top and the pelvis at the bottom.

The vertebra is composed of 7 cervical vertebrae, 12 thoracic vertebrae, 5 lumbar vertebrae, 5 sacral vertebrae (sacrum), and 3 to 5 coccygeal vertebrae (coccyx). In adults, the five sacral vertebrae fuse into one sacrum, and the three to five coccygeal vertebrae fuse into one coccyx.

Spinal fusion is a method of treating serious spinal diseases that last for a long time. In spinal fusion surgery, an intervertebral disk is removed, and a cage is inserted as a substitute between adjacent vertebral bodies to join the adjacent vertebral bodies together.

The spinal fusion methods for the lumbar spine may be classified, depending on the insertion direction of a cage, into posterior lumbar interbody fusion (PLIF), transforaminal lumbar interbody fusion (TLIF), lateral lumbar interbody fusion (LLIF), oblique lumbar interbody fusion (OLIF), anterior lumbar interbody fusion (ALIF), etc.

Posterior lumbar interbody fusion (PLIF) is a method of making an incision along the midline of the lumbar spine to entirely expose a vertebra, removing a posterior portion of the vertebra, removing a disk, and inserting PLIF cages.

Posterior lumbar interbody fusion (PLIF) is the oldest method among lumbar interbody fusion methods, and is necessary when fusing two or three vertebrae together. However, posterior lumbar interbody fusion (PLIF) has disadvantages such as a high possibility of adhesions at the nerves, ligaments, and muscles due to surgical procedures, a long recovery time due to a large incision area, and serious sequelae in some patients.

PLIF cages, a pair of small cages configured to be arranged at the left and right sides, are smallest among all the cages used for spinal fusion.

Transforaminal lumbar interbody fusion (TLIF) is a surgical method in which small incisions are made along both sides of a spine muscle to minimally expose the body of a vertebra, and then while removing a portion of the vertebra to expose a neural foramen, a TLIF cage is inserted instead of a disk. This surgical technique is advantageous in terms of less bleeding and a short operation time and is suitable for one joint, but PLIF is required when treating multiple sites. Most TLIF cages are arc shaped, and thus the convex portion of a TLIF cage is oriented toward the abdomen by inserting and rotating the TLIF cage between vertebral bodies. TLIF cages are larger than PLIF cages, but the supporting areas of TLIF cages are smaller than those of LLIF cages or ALIF cages, which will be mentioned later.

Anterior lumbar interbody fusion (ALIF) has several advantages, such as quick recovery from surgery and a low possibility of adhesions. However, ALIF requires a highly advanced skill in making an incision in the anterior (abdomen) and accessing the spine while dislodging the internal organs. ALIF cages have an advantage of having the largest support areas among all interbody fusion cages.

Lateral lumbar interbody fusion (LLIF) was developed to overcome the disadvantages of ALIF, PLIF and TLIF. LLIF makes an incision in the side and thus can more widely expand the space of a stenosis between the vertebrae than other surgical methods which make an incision in the back, and involves little damage to the surrounding tissues. However, the psoas muscle and peritoneum are on the way of accessing the site, and thus a mistake during the operation may cause numbness in the thigh, etc. LLIF cages are smaller than ALIF cages, but smaller than PLIF cages or TLIF cages.

Oblique lumbar interbody fusion (OLIF) or anterior to psoas (ATP) fusion is a safer and more effective method than LLIF. OLIF accesses the site from the side in an oblique direction, and is operable between the fourth lumbar vertebra L4 and the fifth lumbar vertebra LS where LLIF can hardly access due to the psoas muscle and peritoneum. Also, OLIF has an advantage of causing significantly less damage to the nerve, which is the problem of LLIF.

Existing spinal fusion cages were formed of one body with the same cross-sectional area or height using a metal material such as titanium or a polymer material such as PEEK. As such, there are a good number of product groups considering the physique, height, race, gender, etc. of patients. In other words, manufacturers have to manufacture at least tens of to as many as hundreds of product groups by combining three variables, width, length and height.

Also, the spaces between the vertebrae of patients do not widen at regular intervals. When the cages are formed of one body, a cage with a proper height should be selected from the existing product groups. Thus, this could not meet each patient's condition perfectly.

Many attempts have been made to solve the problem, and spinal fusion cages capable of adjusting height were developed.

U.S. Pat. No. 6,176,882 discloses a height-adjustable cage. The cage of U.S. Pat. No. 6,176,882 comprises walls in a shape of a square box having an open top and an open bottom, an engagement member moving vertically inside the walls, a pair of wedge members pushing out the engagement member, and an adjusting element screw-coupled to the pair of wedge members and adjusting the distance between the pair of wedge members. According to U.S. Pat. No. 6,176,882, the engagement member and the wedge members are not connected with each other, but are simply constrained by the walls of a box shape, and thus the engagement member shakes.

U.S. Pat. No. 9,034,041, claim 1 of D4, discloses comprising a body assembly, an upper support member 718, and a lower support member 720, wherein the body assembly comprises a first portion 712 and a second portion 714, the first portion 712 and the second portion 714 being movable by a control member along a longitudinal axis. The distance between the upper support member 718 and the lower support member 720 is controlled by a first upper pair of retaining members and a second upper pair of retaining members. As such, U.S. Pat. No. 9,034,041 does not comprise an element for directly guiding the movements of the upper support member 718 and the lower support member 720 relative to each other, and thus the body assembly, and the upper support member 718 and the lower support member 720 shake relative to each other.

US2017-0258605A in FIGS. 26 to 29 discloses a holder 400 for a height-adjustable cage. US2017-0258605 relates to a method of fixing the cage 300 by inserting or protruding a plurality of arms 402 into or from a sleeve 410 and mounting protrusions 404 at the ends of the arms 402 to recesses 320 of the cage 300. However, such method has a problem that the holder 400 may not be separated from the implant 302 because the arms 420 having elasticity may expand due to repeated use or the surrounding muscle may disturb the operation at the surgical site.

### PRIOR ART REFERENCE

### Patent Document

(Patent Document 1) U.S. Pat. No. 6,176,882
(Patent Document 2) U.S. Pat. No. 9,034,041
(Patent Document 3) US2017-0258605A

### DETAILED DESCRIPTION OF INVENTION

### Technical Task

An object of the present invention derived for solving the above-mentioned problems is to provide a spinal fusion cage which is inserted between vertebral bodies in a state where the cage has the lowest height, is height-adjustable in the inserted state, and can stably support the movement of a pair of end plates.

### Means for Solving Technical Task

In order to achieve the above object, the present invention provides a spinal fusion cage, comprising: a first end plate and a second end plate which are in contact with adjacent vertebral bodies; a distal movable block connected to be movable relative to distal portions of the first end plate and the second end plate; a proximal movable block connected to be movable relative to proximal portions of the first end plate and the second end plate; an adjustment member capable of adjusting the distance between the distal movable block and the proximal movable block by adjusting the distance between the proximal movable block and the distal movable block by rotation; and a vertical guide portion disposed in the first end plate and second end plate, to support a load in the longitudinal direction or width direction of the first end plate and the second end plate, wherein the vertical guide portion has a first vertical guide formed in the thickness direction of the first end plate or the second end plate, and a second vertical guide formed in the thickness direction of the first end plate or the second end plate to be slidable with the first vertical guide, wherein two pairs of the vertical guides are arranged, one pair of the vertical guides is arranged on one side of the first end plate and the second end plate in the width direction, and the other pair of the vertical guides is arranged on the other side of the first end plate and the second end plate in the width direction.

A block slider is formed on the distal movable block and the proximal movable block, and a plate slider slidable relative to the block slider is formed on the plate slope portion.

The adjustment member has a threaded portion screw-coupled to the distal movable block on an end thereof and a rotation support place fixed to be rotatable relative to the proximal movable block on the other end thereof, wherein a rotation support member is positioned in the rotation support place through the proximal movable block.

The first vertical guide is a pillar protruding from the first or second end plate in the thickness direction, and the second vertical guide has a channel surrounding part of the outer surface of the pillar.

The channel is formed by a first recessed portion arranged concavely in the first or second end plate in the width direction and a second recessed portion formed by an extension portion protruding from the recessed portion in the thickness direction of the first or second end plate.

An accommodation groove accommodating the extension portion is formed around the pillar.

The first recessed portion is formed concavely by a first recessed wall disposed on the side of the proximal portion, a second recessed wall spaced part from the first recessed wall and disposed on the side of the distal portion, and a third recessed wall connecting the first recessed wall and second recessed wall.

The thickness of the pillar in the width direction is the same as the depth of the first recessed portion in the width direction, and the thickness of the pillar in the longitudinal direction is the same as the distance between the first recessed wall and the second recessed wall in the longitudinal direction.

The second recessed portion is formed by an extension portion including a first extension wall protruding from the first or second end plate in the thickness direction and disposed on the side of the proximal portion, a second extension wall protruding from the first or second end plate in the thickness direction and disposed on the side of the distal portion, and a third extension wall protruding from the first or second end plate in the thickness direction and connecting the first extension wall and the second extension wall, wherein the first extension wall and the first recessed wall form a plane, the second extension wall and the second recessed wall form a plane, and the third extension wall and the third recessed wall form a plane.

The depth of the second recessed portion in the width direction is smaller than the depth of the first recessed portion in the width direction.

A guide groove guiding the insertion of the pillar is formed around the first recessed portion.

The sum of the thicknesses of the first extension wall and the second extension wall in the longitudinal direction is greater than or equal to the thickness of the pillar in the longitudinal direction.

The thickness of the third extension wall in the width direction is the same as the thickness of the pillar in the width direction.

An expansion groove is arranged on the bottom surfaces facing each other in the first and second end plates.

A through hole is formed in the adjustment member, a communication hole in communication with the through hole is formed in the distal movable block, and a discharge hole in communication with the communication hole is formed on the side portion of the distal movable block.

### Effect of Invention

According to the present invention, cages having heights within a given range can be replaced with one cage. Therefore, manufacturers may reduce the number of product groups and the amount of inventory. Additionally, since the height of the cage is linearly adjustable according to the distance between the vertebral bodies of patients unlike conventional cages having heights preset at predetermined intervals, surgery may be performed at an optimal height according to the conditions of patients.

Also, since the cage is inserted at the lowest height, the burden of separately producing test inserts according to the existing proper intervertebral spacing may be reduced, and the effort of securing an insertion space while inserting a plurality of test inserts sequentially may be reduced from the doctor's point of view.

### BRIEF DESCRIPTION OF DRAWINGS

FIG. 1 is a perspective view of a spinal fusion cage in the lowest state according to an embodiment of the present invention;

FIG. 2 is a perspective view illustrating the spinal fusion cage of FIG. 1 in the highest state;

FIG. 3 is a top exploded perspective view illustrating the spinal fusion cage of FIG. 1;

FIG. 4 is a bottom exploded perspective view illustrating the spinal fusion cage of FIG. 1;

FIG. 5 is a top perspective view illustrating the first end plate of the spinal fusion cage of FIG. 1;

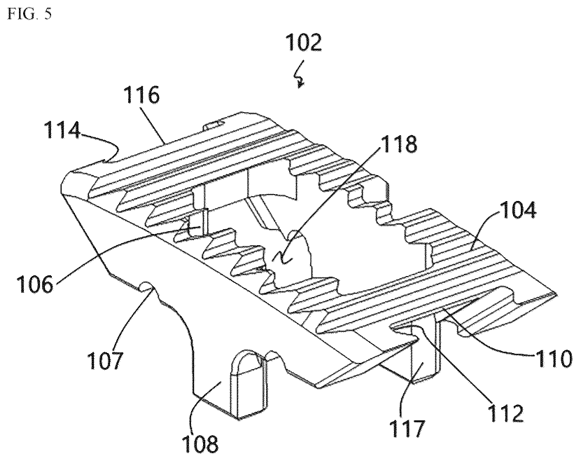

FIG. 6 is a bottom perspective view illustrating the first end plate of the spinal fusion cage of FIG. 1;

FIG. 7 is a top perspective view illustrating the second end plate of the spinal fusion cage of FIG. 1;

FIG. 8 is a bottom perspective view illustrating the second end plate of the spinal fusion cage of FIG. 1;

FIG. 9 is a partial enlarged view of FIG. 8;

FIG. 10 is a top perspective view illustrating the distal movable block of the spinal fusion cage of FIG. 1;

FIG. 11 is a bottom perspective view illustrating the distal movable block of the spinal fusion cage of FIG. 1;

FIG. 12 is a cross-sectional view illustrating the distal movable block of the spinal fusion cage of FIG. 1;

FIG. 13 is a top perspective view illustrating the proximal movable block of the spinal fusion cage of FIG. 1;

FIG. 14 is a bottom perspective view illustrating the proximal movable block of the spinal fusion cage of FIG. 1;

FIG. 15 is a perspective view illustrating the adjustment member of the spinal fusion cage of FIG. 1; and

FIG. 16 is a cross-sectional view illustrating the adjustment member of the spinal fusion cage of FIG. 1.

### BEST MODE FOR CARRYING OUT THE INVENTION

Hereinafter, preferred embodiments of the present disclosure will be described with reference to the accompanying drawings. In the drawings, the same elements may be denoted with the same reference numerals even though the elements are shown in different drawings, and detailed descriptions related to well-known functions or configurations will be ruled out in order not to unnecessarily obscure subject matters of the present disclosure.

The directions as used herein will be defined. A distal direction means a direction into which the spinal fusion cage is inserted, and a proximal direction means a direction opposite to the distal direction. A longitudinal direction means a virtual linear direction connecting the distal direction and proximal direction. A thickness direction means a thickness direction of the end plate, i.e., a virtual linear direction toward the vertebrae above and below. A width direction, a direction perpendicular to both the longitudinal direction and thickness direction, means a horizontal direction of the end plate.

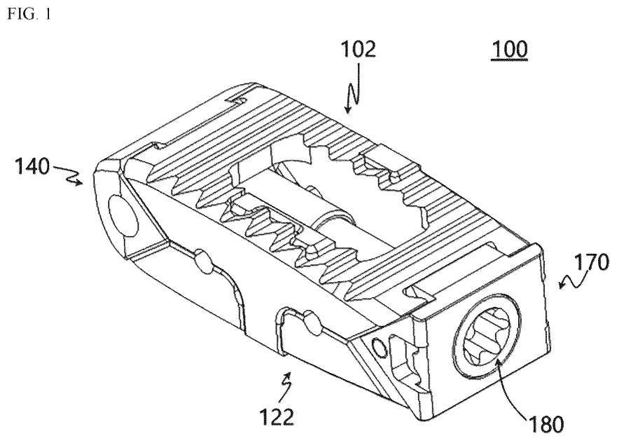

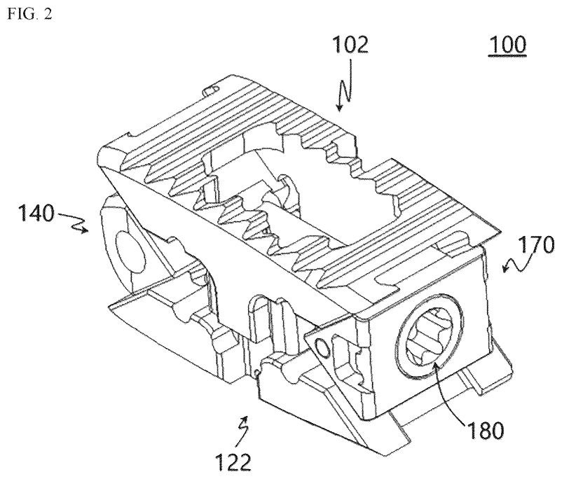

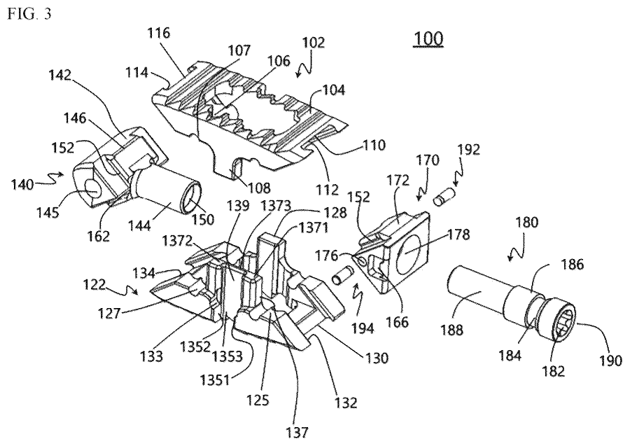

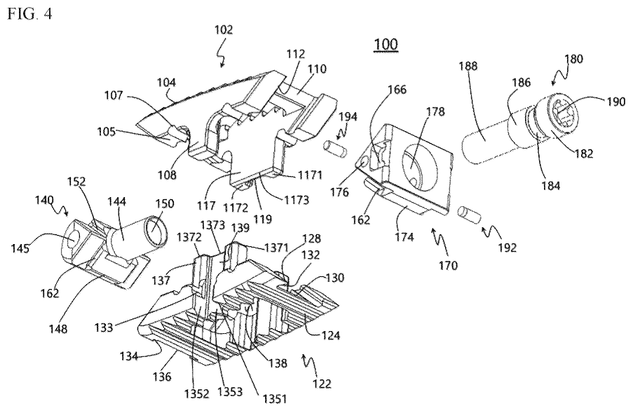

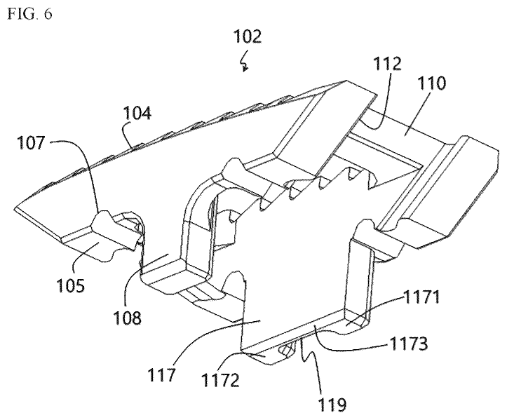

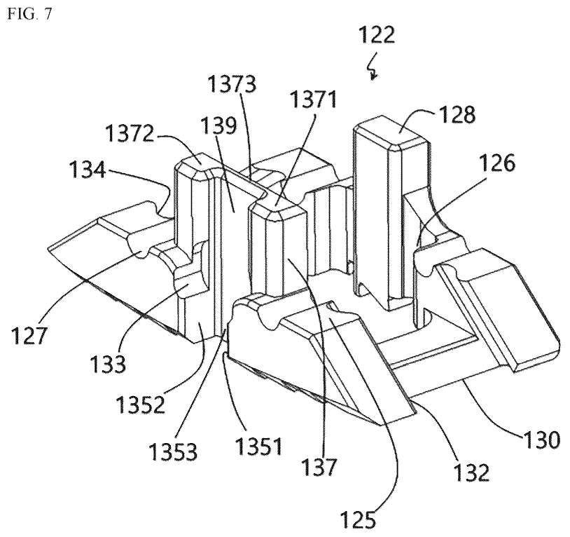

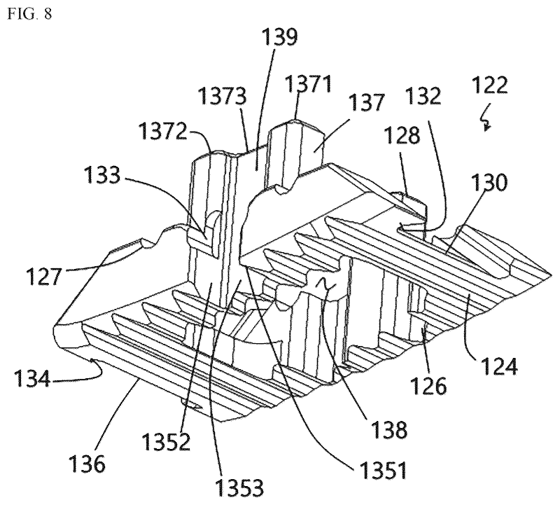

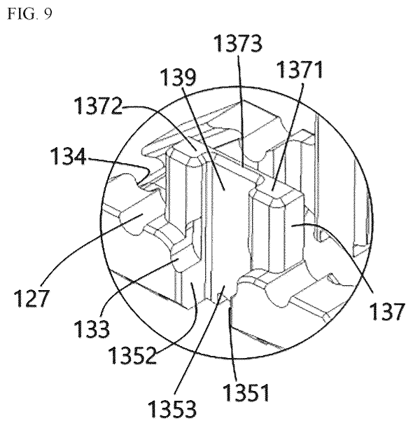

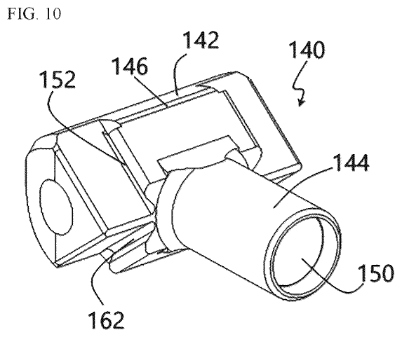

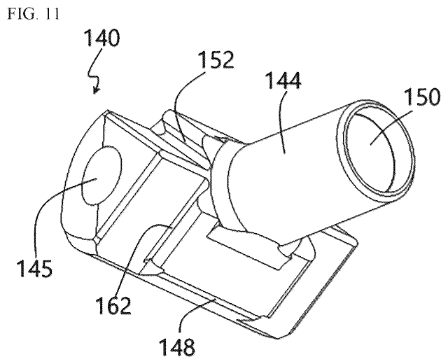

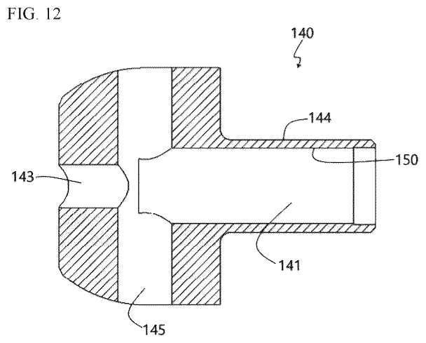

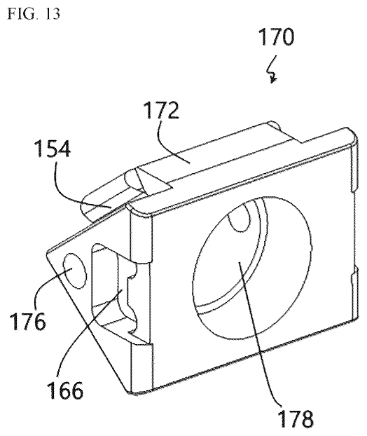

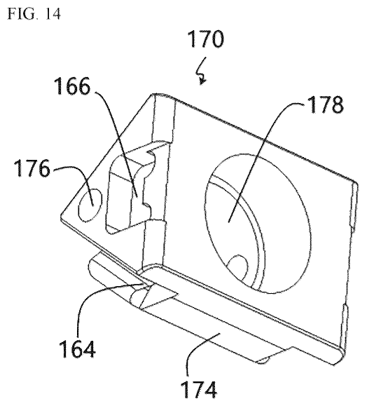

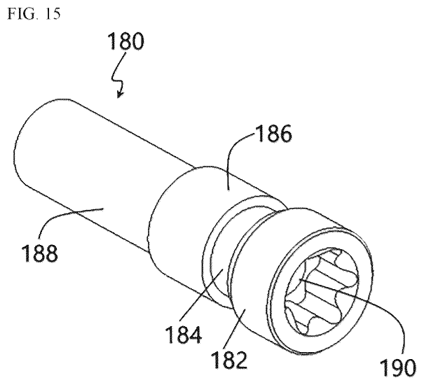

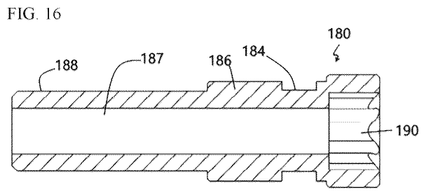

FIGS. 1 to 4 illustrate the entire spinal fusion cage 100 according to an embodiment, and FIGS. 6 to 16 illustrate each element of the spinal fusion cage 100.

The spinal fusion cage 100 includes a first end plate 102 and a second end plate 122 which face each other in a vertical direction, a distal movable block 140 and a proximal movable block 170 which are arranged between the first end plate 102 and the second end plate 122 to move along the distance between the first end plate 102 and the second end plate 122, and an adjustment member 180 which is capable of adjusting the distance between the distal movable block 140 and the proximal movable block 170 by adjusting the distance between the proximal movable block 170 and the distal movable block 140 by rotation. Further, the spinal fusion cage 100 includes a vertical guide portion which is disposed in the first end plate 102 and the second end plate 122 to support a load in the longitudinal direction or width direction of the first end plate 102 and the second end plate 122.

The first end plate 102 and the second end plate 122 include a first end plate body 104 and a second end plate body 124 which are in contact with vertebral bodies. The first end plate body 104 and the second end plate body 124 may have tooth-shaped protrusions to prevent separation from the vertebral bodies. Additionally, a first window 118 and a second window 138 are respectively formed in a center portion of the first end plate body 104 and a center portion of the second end plate body 124 such that a bone graft may be inserted therethrough.

First block places 110, 116 are formed on both ends of the first end plate body 104 in the longitudinal direction, and first plate rails 112, 114 are formed in the first block places 110, 116, respectively. Part of the distal movable block 140 and the proximal movable block 170 may be accommodated in the first block places 110, 116. For the first plate rails 112, 114, a pair of rails facing each other is arranged on both sides of the first block places 110, 116. The first plate rail 112 formed in the distal direction and the first plate rail 114 formed in the proximal direction are formed to be sloped in a manner approaching the center of the first end plate body 104 as they go away from the surface of the first end plate body 104 in the thickness direction.

Similarly, second block places 130, 136 are formed on both ends of the second end plate body 124 in the longitudinal direction, and second plate rails 132, 134 are formed in the second block places 130, 136, respectively. Part of the distal movable block 140 and the proximal movable block 170 may be accommodated in the second block places 130, 136. For the second plate rails 132, 134, a pair of rails facing each other is arranged on both sides of the second block places 130, 136. The second plate rail 132 formed in the distal direction and the second plate rail 134 formed in the proximal direction are formed to be sloped in a manner approaching the center of the second end plate body 124 as they go away from the surf ace of the second end plate body 124 in the thickness direction.

Expansion grooves 107, 127 are arranged on the bottom surfaces 105, 125 facing each other in the first and second end plates 102, 122. The expansion grooves 107, 127 are elongated to communicate with the centers of the first and second end plates 102, 122 in the width direction. In the present embodiment, the expansion grooves have a substantially semicylindrical shape in the width direction such that the semicylindrical shape becomes a cylindrical shape when the first and second end plates 102, 122 are moved maximally close to each other. The expansion grooves 107, 127 communicate the bone graft inside and outside the spinal fusion cage 100 with each other when the first and second end plates 102, 122 are moved close to each other, and further expand the space where the bone graft is filled when the first and second end plates 102, 122 are moved away from each other.

The distal movable block 140 has a streamlined shape with an insertion portion 142 protruding for ease of insertion between the vertebral bodies in the distal direction. The distal movable block 140 has a connection portion 144 elongated in the proximal direction, and a connection threaded portion 150 having a screw thread is formed inside the connection portion 144. The distal movable block 140 has a first block protrusion 146 to correspond to the first block place 116 of the first end plate 102 and has a second block protrusion 148 to correspond to the second block place 136 of the second end plate 122. A first block rail 152 corresponding to the first plate rail 114 is formed around the first block protrusion 146. A second block rail 148 corresponding to the second plate rail 136 is formed around the second block protrusion 148.

The proximal movable block 170 has an adjustment member hole 178 to support the adjustment member 180 to be rotatable thereinside. The proximal movable block 170 has a first block protrusion 172 to correspond to the first block place 110 of the first end plate 102 and has a second block protrusion 174 to correspond to the second block place 130 of the second end plate 122. A first block rail 154 corresponding to the first plate rail 112 is formed around the first block protrusion 172. A second block rail 164 corresponding to the second plate rail 132 is formed around the second block protrusion 174. A pinhole 176 accommodating pin members 192, 194 is formed on the side portion of the proximal movable block 170. In addition, a fastening portion 166 is formed on the side portion of the proximal movable block 170, to hold the spinal fusion cage 100 by a tool.

The distal movable block 140 and the proximal movable block 170 have a substantially wedge shape, and are configured to push or support the first end plate 102 and the second end plate 122 to lift or lower the same.

The adjustment member 180 may have a substantially bolt shape. That is, the adjustment member 180 has a head 182 and an adjustment threaded portion 188. The head 182 is disposed in the opening formed in the proximal direction of the adjustment member hole 178, and the adjustment threaded portion 188 passes through the adjustment member hole 178 and is screw-coupled to the connection threaded portion 150 of the connection portion 144. A tool place 190 connectable with a tool that is not shown is formed in the head 182. In addition, a rotation support portion 186 positioned between the head 182 and the adjustment member hole 178 supports rotation, while being in contact with the inner wall of the adjustment member hole 178. A pin place 184 is formed around the rotation support portion 186 such that the ends of the pin members 192, 194 inserted through the pinhole 176 of the proximal movable block 170 are placed therein. As a result, the adjustment member 180 is rotatable in position.

In order to deliver a bone graft, a through hole 187 is formed in the adjustment member 180 in the longitudinal direction, a communication hole 141 in communication with the through hole 187 is formed in the distal movable block 140, and a discharge hole 145 in communication with the communication hole 141 is formed on both sides of the side portion of the distal movable block 140. As a result, when a bone graft is injected through the through hole 187 of the adjustment member 180, the material passes through the communication hole 141 and is discharged to the discharge hole 145, and thus it is possible to supply the bone graft around the distal movable block 140. In addition, when a guide hole 143 is formed in the distal movable block 140 in the longitudinal direction to communicate with the communication hole 141, a guide wire can pass through the spinal fusion cage 100 using the guide hole 143, the communication hole 141, and the through hole 187. This may help insert the spinal fusion cage 100 during surgery. Here, it is preferable to make the cross-sectional area of the guide hole 143 smaller than that of the discharge hole 145, such that a larger amount of the bone graft is discharged to the discharge hole 145 than to the guide hole 143.

The vertical guide portion includes a first vertical guide formed in the thickness direction of the first end plate 102 or the second end plate 122 and a second vertical guide formed in the thickness direction of the first end plate 102 or the second end plate 122 to be slidable with the first vertical guide.

Two pairs of the vertical guides are arranged, one pair of which is arranged on one side of the first end plate 102 and the second end plate 122 in the width direction, and the other pair of which is arranged on the other side of the first end plate 102 and the second end plate 122 in the width direction. The following three cases are possible. First, the first vertical guide is installed on both sides of the first end plate 102, and the second vertical guide is installed on both sides of the second end plate 122. Second, the second vertical guide is installed on both sides of the first end plate 102, and the first vertical guide is installed on both sides of the second end plate 122. Third, the first vertical guide and the second vertical guide are installed on both sides of the first end plate 102, respectively, and the second vertical guide and the first vertical guide are installed on both sides of the second end plate 122 to correspond to those of the first end plate 102. Hereinafter, the third case is described as an example. Here, this is the same for the case where the first vertical guide and the second vertical guide of the first end plate 102 and the second end plate 122 are exchanged with each other to the left and right sides, and thus the description thereon is omitted. In addition, the description on the first and second cases is omitted for the same reasons.

The first vertical guide is a pillar 108, 128 protruding from the first or second end plate 102, 122 in the thickness direction, and the second vertical guide has a channel 119, 139 surrounding part of the outer surface of the pillar 108, 128.

The channel 119, 139 is formed by a first recessed portion concavely arranged in the first end plate 102 or the second end plate 122 in the width direction and a second recessed portion formed by an extension portion 117, 137 protruding from the recessed portion in the thickness direction of the first end plate 102 or the second end plate 122. For the sake of convenience in explanation, the first end plate 102 has the first pillar 108, first extension portion 117 and first channel 119, and the second end plate 122 has the second pillar 128, second extension portion 137 and second channel 139.

The first recessed portion is concavely formed by a first recessed wall 1351 disposed on the side of the proximal portion, a second recessed wall 1352 spaced apart from the first recessed wall 1351 and disposed on the side of the distal portion and a third recessed wall 1353 connecting the first recessed wall 1351 and the second recessed wall 1352. The first recessed portion is shown only in the second end plate 122 in the drawings, and thus the description on the first recessed portion of the first end plate 102 is omitted.

The second recessed portion is formed by first and second extension portions 117, 137 that include a first extension wall 1171, 1371 protruding from the first end plate 102 or the second end plate 122 in the thickness direction and disposed on the side of the proximal portion, a second extension wall 1172, 1372 protruding from the first end plate 102 or the second end plate 122 in the thickness direction and disposed on the side of the distal portion, and a third extension wall 1173, 1373 protruding from the first end plate 102 or the second end plate 122 in the thickness direction and connecting the first extension wall 1171, 1371 and the second extension wall 1172, 1372.

Accordingly, the first recessed portion and the second recessed portion form a substantially U shape. The first extension wall 1171, 1371 and the first recessed wall 1351 form a plane, the second extension wall 1172, 1372 and the second recessed wall 1352 form a plane, and the third extension wall 1173, 1373 and the third recessed wall 1353 form a plane. As a result, the first recessed portion and the second recessed portion form the first and second channels 119, 139 as a whole, and the first and second pillars 108, 128 are slidable relative to the first and second channels 119, 139.

The depth of the second recessed portion in the width direction is smaller than the depth of the first recessed portion in the width direction. Here, the depth of the first and second pillars 108, 128 in the width direction is the same as the depth of the first recessed portion in the width direction, and the thickness of the first and second pillars 108, 128 in the longitudinal direction is the same as the distance between the first recessed wall 1351 and the second recessed wall 1352 in the longitudinal direction.

Accordingly, when the first and second pillars 108, 128 are inserted into the first and second channels 119, 139, the first recessed portion surrounds two surfaces in the longitudinal direction and one surface in the width direction of the first and second pillars 108, 128, whereas the second recessed portion surrounds the whole of one surface in the width direction but part of two surfaces in the longitudinal direction of the first and second pillars 108, 128.

In addition, in order to support a force applied in the longitudinal direction, preferably, the sum of the thicknesses of the first extension wall 1171, 13 71 and the second extension wall 1172, 1372 in the longitudinal direction is greater than or equal to the thickness of the pillar in the longitudinal direction. In order to maximize a force applied in the width direction, preferably, the thickness of the third extension wall 1173, 1373 in the width direction is the same as the thickness of the pillar 108, 128 in the width direction.

A guide groove 113, 133 for guiding the insertion of the pillar 108, 128 is formed around the first recessed portion, such that the pillar 108, 128 is naturally guided to the side of the first recessed portion when the spinal fusion cage 100 is assembled or the height thereof is lowered after being raised.

The spinal fusion cage 100 is configured as described above. The proximal movable block 170 and the distal movable block 140 are moved close to each other by inserting a tool such as a screwdriver into a tool groove 190 and rotating the tool in one direction. As a result, the first end plate 102 and the second end plate 122 can be moved away from each other. Similarly, the proximal movable block 170 and the distal movable block 140 are moved away from each other by inserting the tool and rotating the same in the other direction. As a result, the first end plate 102 and the second end plate 122 can be moved close to each other.

While preferred embodiments of the present disclosure have been described as above, it will be understood by those of ordinary skill in the art that various changes in form and details may be made therein without departing from the spirit and scope of the present disclosure as defined by the following claims.

### INDUSTRIAL APPLICABILITY

According to the present invention, one cage may be used for a given height range, thereby reducing the burden of inventory and production. Additionally, repetitive tasks during surgery are reduced, thereby reducing burdens on surgeons. In addition, the operation time and the amount of bleeding may be reduced, and thus the recovery time of patients may be greatly reduced. Therefore, the present disclosure may be widely used in related fields.

### DESCRIPTION OF REFERENCE NUMERALS

100: spinal fusion cage
102: first end plate
104: first end plate body
105: first bottom surface
106: first accommodation groove
107: first expansion groove
108: first pillar
110, 116: first block place
112, 114: first plate rail
117: first extension portion
118: first window
119: first channel
122: second end plate
124: second end plate body
126: second accommodation groove
127: second expansion groove
128: second pillar
130, 136: second block place
132, 134: second plate rail
137: second extension portion
138: second window
139: second channel
140: distal movable block
141: communication hole
142: insertion portion
143: guide hole
144: connection portion
145: discharge hole
146, 172: first block protrusion
148, 174: second block protrusion
150: connection threaded portion
152, 154: first block rail
162, 164: second block rail
166, 266: fastening portion
170: proximal movable block
176: pinhole
178: adjustment member hole
180: adjustment member
182: head
184: pin place
186: rotation support portion
188: adjustment threaded portion
190: tool place
192, 194: pin member
1371: first extension wall
1372: second extension wall
1373: third extension wall
1351: first recessed wall
1352: second recessed wall
1353: third recessed wall

What is claimed is:

1. A spinal fusion cage, comprising:
a first end plate and a second end plate which are in contact with adjacent vertebral bodies;
a distal movable block connected to be movable relative to distal portions of the first end plate and the second end plate;
a proximal movable block connected to be movable relative to proximal portions of the first end plate and the second end plate;
an adjustment member capable of adjusting the distance between the distal movable block and the proximal movable block by adjusting the distance between the proximal movable block and the distal movable block by rotation; and
a vertical guide portion disposed in the first end plate and second end plate, to support a load in the longitudinal direction or width direction of the first end plate and the second end plate,
wherein the vertical guide portion has a first vertical guide formed in the thickness direction of the first end plate or the second end plate, and a second vertical guide formed in the thickness direction of the first end plate or the second end plate to be slidable with the first vertical guide,
wherein two pairs of the vertical guides are arranged,
one pair of the vertical guides is arranged on one side of the first end plate and the second end plate in the width direction, and
the other pair of the vertical guides is arranged on the other side of the first end plate and the second end plate in the width direction.

2. The spinal fusion cage of claim 1, wherein a block slider is formed on the distal movable block and the proximal movable block, and a plate slider slidable relative to the block slider is formed on the plate slope portion.

3. The spinal fusion cage of claim 1, wherein the adjustment member has a threaded portion screw-coupled to the distal movable block on an end thereof and a rotation support place fixed to be rotatable relative to the proximal movable block on the other end thereof, wherein a rotation support member is positioned in the rotation support place through the proximal movable block.

4. The spinal fusion cage of claim 1, wherein the first vertical guide is a pillar protruding from the first or second end plate in the thickness direction, and the second vertical guide has a channel surrounding part of the outer surface of the pillar.

5. The spinal fusion cage of claim 4, wherein the channel is formed by a first recessed portion arranged concavely in the first or second end plate in the width direction and a second recessed portion formed by an extension portion protruding from the recessed portion in the thickness direction of the first or second end plate.

6. The spinal fusion cage of claim 5, wherein an accommodation groove accommodating the extension portion is formed around the pillar.

7. The spinal fusion cage of claim 5, wherein the first recessed portion is formed concavely by a first recessed wall disposed on the side of the proximal portion, a second recessed wall spaced part from the first recessed wall and disposed on the side of the distal portion, and a third recessed wall connecting the first recessed wall and second recessed wall.

8. The spinal fusion cage of claim 7, wherein the thickness of the pillar in the width direction is the same as the depth of the first recessed portion in the width direction, and the thickness of the pillar in the longitudinal direction is the same as the distance between the first recessed wall and the second recessed wall in the longitudinal direction.

9. The spinal fusion cage of claim 7, wherein the second recessed portion is formed by an extension portion including a first extension wall protruding from the first or second end plate in the thickness direction and disposed on the side of the proximal portion, a second extension wall protruding from the first or second end plate in the thickness direction and disposed on the side of the distal portion, and a third extension wall protruding from the first or second end plate in the thickness direction and connecting the first extension wall and the second extension wall,
wherein the first extension wall and the first recessed wall form a plane, the second extension wall and the second recessed wall form a plane, and the third extension wall and the third recessed wall form a plane.

10. The spinal fusion cage of claim 9, wherein the depth of the second recessed portion in the width direction is smaller than the depth of the first recessed portion in the width direction.

11. The spinal fusion cage of claim 7, wherein a guide groove guiding the insertion of the pillar is formed around the first recessed portion.

12. The spinal fusion cage of claim 9, wherein the sum of the thicknesses of the first extension wall and the second extension wall in the longitudinal direction is greater than or equal to the thickness of the pillar in the longitudinal direction.

13. The spinal fusion cage of claim 9, wherein the thickness of the third extension wall in the width direction is the same as the thickness of the pillar in the width direction.

14. The spinal fusion cage of claim 1, wherein an expansion groove is arranged on the bottom surfaces facing each other in the first and second end plates.

15. The spinal fusion cage of claim 1, wherein a through hole is formed in the adjustment member, a communication hole in communication with the through hole is formed in the distal movable block, and a discharge hole in communication with the communication hole is formed on the side portion of the distal movable block.
